(19) United States
(12) Patent Application Publication
(10) Pub. No.: US 2021/0162180 A1
(43) Pub. Date: Jun. 3, 2021

(54) CATHETER ADAPTED BRIEFS

(71) Applicant: Meyer David GERSHBAUM, Woodmere, NY (US)

(72) Inventor: Meyer David GERSHBAUM, Woodmere, NY (US)

(21) Appl. No.: 16/699,021

(22) Filed: Nov. 28, 2019

(51) Int. Cl. A61M 25/02; A61M 25/00; A41B 9/02

(52) U.S. Cl. CPC A61M 25/02; A61M 25/0017; A41B 9/026; A61M 2025/0206; A41B 2400/34; A61M 2210/1096; A41B 2300/32; A41B 2300/324; A61M 2025/024

(57) ABSTRACT

Catheter-adapted briefs operative to stabilize an indwelling urethra catheter in a position of alignment with the natural orientation of the male urethral orifice to minimize catheter discomfort, prevent urine leakage, tissue damage, and reduce the frequency of urinary track infection.

### BACKGROUND OF THE INVENTION

Indwelling urethral catheters of male users typically drain into a urinary pouch attached to the leg. Since the urinary pouch is disposed in non-alignment with the anatomical center, the catheter may place pressure on the penis and urethral orifice thereby creating catheter discomfort, urine leakage, increased incidence of urinary tract infection, and penile erosion.

Taping the catheter to the wearer's thigh or abdomen is a frequently used method for stabilization. However, tape can irritate the skin and can loosen quickly thereby rendering the stabilization non-effective.

Elastic catheter straps having velcro strip are also used to secure the catheter to one's thigh. This addresses the skin irritation problem associated with taping; however, it suffers from inadvertent loosening or slippage and can also cause constriction of the thigh if applied too tightly leading to discomfort, skin breakdown, and swelling.

Strap free adhesive anchors have a locking device and are used as an anchoring device for the catheter. They usually have an adhesive backing that is applied directly to the skin of the thigh or lower abdomen. Here too, though, the adhesive can cause skin irritation and the anchors tend to loosen and fall off the skin.

Therefore, there is a need for a long-term catheter securing device that is comfortable, does not cause skin irritation, and does not constrict the during use.

### SUMMARY OF THE INVENTION

There is provided according to the teachings of the present invention, a catheter-adapted briefs including: a pair of briefs having a catheter port disposed in vertical alignment with the male urethral orifice when the briefs are worn; and a catheter clamp connected to the briefs, the clamp operative to secure an implanted urethral catheter passing through the catheter port in communication with a drainage bag, such that force transmission from the bag through the catheter is halted by the clamp when securing the catheter.

According to a further feature of the present invention, the catheter port is stich reinforced.

According to a further feature of the present invention, the briefs include a functional fly.

According to a further feature of the present invention, the functional fly extends through a waistband band of the briefs so as to enable a catheter wearer to put on the briefs without disconnecting the catheter from the drainage bag.

According to a further feature of the present invention, the functional fly has a fastener chosen from the group consisting of a hook-and-eye fasteners, zipper, or magnetic fastener.

According to a further feature of the present invention, the catheter clamp includes one or more catheter channels, each of the channels having a width matching an outer diameter of a catheter.

According to a further feature of the present invention, the catheter clamp is releasably attachable to the briefs.

According to a further feature of the present invention, the catheter clamp includes rotatable platform operative to rotate channel orientation.

According to a further feature of the present invention, the catheter clamp is implemented from a polymeric material.

According to a further feature of the present invention, there is also provided a liquid absorbent lining.

According to a further feature of the present invention, the lining is implemented with anti-bacterial material.

According to a further feature of the present invention, the lining includes bamboo fiber.

According to a further feature of the present invention, the lining includes a copper-based material.

### BRIEF DESCRIPTION OF THE DRAWINGS

The subject matter regarded as the invention is particularly pointed out and distinctly claimed in the concluding portion of the specification. The invention is best understood in view of the accompanying drawings in which:

FIGS. 1A and 1B are schematic front and rear views, respectively, of catheter-adapted briefs, according to an embodiment;

FIG. 2A is a schematic front view of the catheter-adapted briefs of FIG. 1A depicting an open functional fly, according to an embodiment;

FIG. 2B depicts a general schematic front view of a second embodiment of the catheter-adapted briefs 5 implemented as a form fitting brief, according to an embodiment;

FIG. 3A is a schematic perspective top view of a catheter clamp attached to the catheter-adapted briefs of FIG. 1, according to an embodiment; and

FIG. 3B is a schematic, front view of the catheter clamp attached to the catheter-adapted briefs of FIG. 1, according to an embodiment.

It will be appreciated that for the sake of clarity, elements shown in the figures may not be drawn to scale and reference numerals may be repeated in different figures to indicate corresponding or analogous elements.

### DETAILED DESCRIPTION OF THE PRESENT INVENTION

In the following detailed description, specific details are set forth in order to facilitate understanding of the invention; however, it should be understood by those skilled in the art that the present invention may be practiced without these specific details. Furthermore, well-known methods, procedures, and components have not been omitted to highlight the invention.

The present invention is a urethral catheter stabilization device, and specifically, is a catheter-adapted underwear, or briefs, operative to comfort to the catheter user, prevention of urine leakage, tissue damage, odor control, and potential reduction of urinary infections. Generally speaking, these favorable conditions are achieved by securing the catheter in a position of alignment with the natural anatomical position of the urethral orifice. This functionality is further complimented with a protective, liquid absorbent, anti-bacterial undergarment.

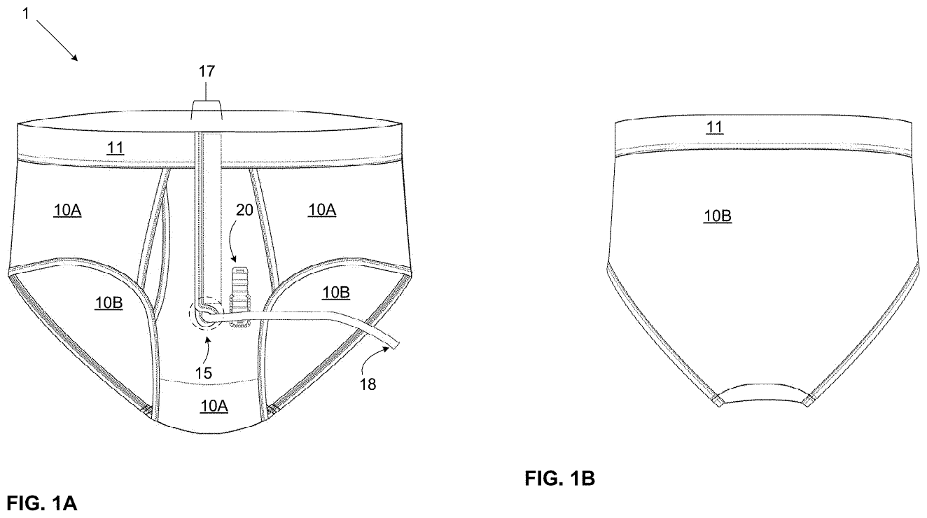

Turning now to the figures, FIGS. 1A and 1B are schematic front and rear views, respectively, of a first embodiment of the catheter-adapted briefs 1 including a front panel 10A, a back panel 10B, a waistband 11, a functional fly 17 traversing waistband 11 to facilitate dressing and undressing with an embedded urethral catheter 18, and a catheter clamp 20 having a plurality of fixation points. Clamp 20 is operative to releasably secure urethral catheter 18 passing through a reinforced catheter port 15. As shown, port 15 is strategically positioned to be in vertical alignment with the natural anatomical position of the urethral orifice of the wearer. This positioning ensures that force applied by embedded urethral catheter 18 to urethral orifice walls is reduced to a minimum and clamp 20 secures catheter 18 in this desirable position.

This catheter securing scheme advantageously maintains proper alignment of the catheter thereby reducing wearer pain and irritation, urine leakage, minimizes the likelihood of penile complications. Furthermore, the proper alignment enables uninterrupted urine flow thereby potentially preventing urinary stasis hence reducing the risk of urinary track infection.

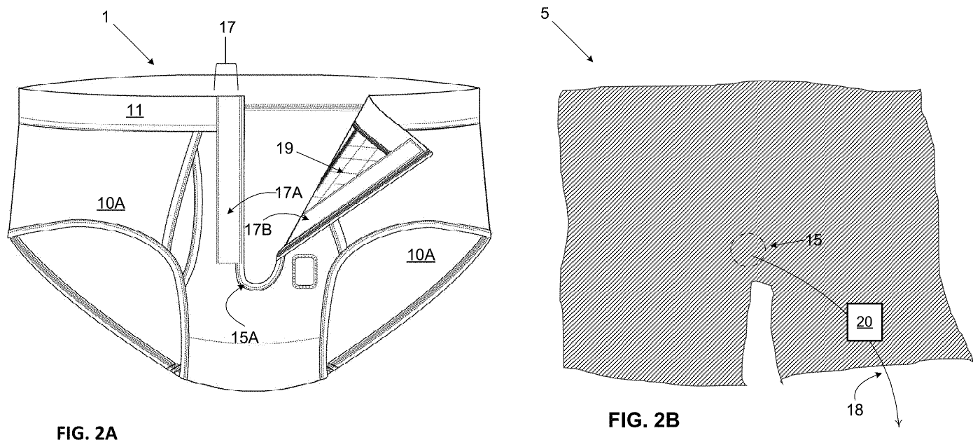

FIG. 2A is a schematic front view of the catheter-adapted briefs of FIG. 1A depicting an open functional fly, according to an embodiment. Fly 17 advantageously traverses waistband 11 to enable a wearer to remove or to put on the briefs without disconnecting catheter 18 from a drain bag (not shown) thereby maintaining a closed drainage system, eliminating urine spill from the bag, and preventing urine contamination.

As shown, fly 17 (shown in FIG. 1) has fasteners 17A and 17B operative to close and open fly 17 during dressing or undressing, respectively. Fly fasteners 17A and 17B in a certain embodiment are implemented as strips of flexible hook-and-loop fasteners commonly known by the generic trademark velcro. In another embodiment, fasteners 17A and 17B are implemented as zipper tape connected by a zipper, whereas in another embodiment fasteners 17A and 17B are implemented as embedded magnets, or rigid hooks and eyes. It should be appreciated that other fasteners types providing such functionality are included within the scope of the present invention. Furthermore, in certain embodiments, various fastener types are employed simultaneously along fly 17, the waistband 11, or both fly and waistband. It should be noted that in a certain embodiment, fly 17 is orientated diagonally relative to the horizontal.

As shown, catheter port walls 15A of port 15 are stich reinforced to facilitate stabilization of catheter 18 and to prevent tearing of panel 10A. In another embodiment, port reinforcement 15A is implemented with metallic, polymeric, or even rubber materials configured to provide free passage of an embedded catheter through the fly and out through waistband 11 during dressing or undressing.

In a certain embodiment front panel 10A is impregnated with antibiotic material like copper-based particles or other antibiotic materials in addition to order absorbent materials. Furthermore, front panel 10A is implemented as a soft, non-irritating material preventing penile irritation, like cotton or other highly absorbent materials also capable of reducing skin irritation from contact with moist or wet material.

As shown, front panel 10A is lined with an interfacing 18 operative to support catheter clamp 20 and disperse tension applied to the clamp through catheter 18. Interfacing 18 is constructed from traditional interfacing materials as is known to those skilled in the art. It should be appreciated that in certain embodiments, bamboo fiber is also used as and interfacing material to provide comfort.

FIG. 2B depicts a second embodiment of catheter-adapted briefs 5 implemented as a form fitting brief to advantageously use the wearer body to support clamp 20. As shown, catheter 18 passes through reinforced port 15, clamp 20 to a catheter bag (not shown) such that catheter tension is directed to clamp 20 supported by the wearer thigh. Form fitted briefs are also constructed from liquid and odor absorbent materials noted above with a mix of Spandex to provide the required elasticity of form fitted briefs.

It should be appreciated that in a certain embodiment, briefs 1 lack a functional fly, whereas in another embodiment briefs 1 have a fly oriented in a non-vertical direction.

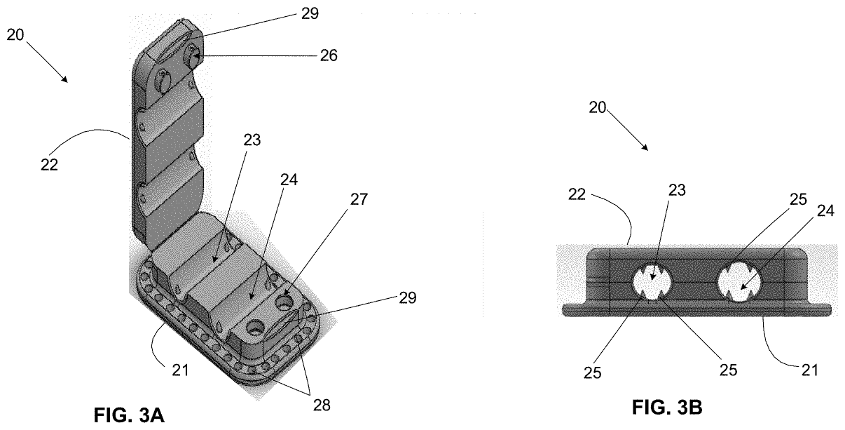

FIG. 3A is a schematic perspective top view schematic of catheter clamp 20 depicted in FIG. 1 and FIG. 3B is a front view of clamp 20, according to an embodiment.

As shown, clamp 20 includes clamp base 21 with a hinged cover 22. Clamp base 21 includes catheter channels 23 and 24, each having a diameter corresponding to an outer diameter of catheter commonly employed in industry. Channels 23 and 24 are fitted with teeth 25 to secure catheter 18 within each channel within clamp cover 22 is closed, in a certain embodiment. In other embodiments, channels 23 and 24 are lined with a compressive material having sufficient stiffness to secure catheters. Example compressive linings include synthetic rubber, stiff foam like GATORFOAM®, or other materials providing analogous functionality. Clamp base 21 also includes engagement bores 27 for releasable engagement of pegs 26 on clamp cover 22 so as to lock cover 22 onto base 21 and secure catheter when closed. Clamp cover 22 ad base 21 both include a release slit 29 that self-aligns during closure and facilitate opening.

Clamp base 21 has a series of eyelets 28 that can be sewed to front panel 10A. It should be appreciated that other connection configurations like clasp, buckles, snaps are employed in certain embodiments to provide releasable connection to briefs 1. Such releasability advantageously enables clamp 20 to be removed during washing. In a certain embodiment clamp 20 is implemented with a single channel.

In a certain embodiment, clamp 20 is constructed from a polymeric a material, whereas in another embodiment entire clamp 20 is constructed from rubber having sufficient stiffness to secure catheter 20. It should be appreciated that clamp 20 is constructed from other material providing such functionality, in yet other embodiments.

It should be appreciated that embodiments formed from combinations of features set forth in separate embodiments are also within the scope of the present invention.

While certain features of the invention have been illustrated and described herein, modifications, substitutions, and equivalents are included within the scope of the invention.

1. Catheter-adapted underpants comprising:
a washable pair of elastic form fitting underpants; and
a catheter clamp sewn to the underpants,
wherein the clamp causes catheter tug to be diverted from a male urethral orifice and distributed to a wearer body part supporting the clamp when the form fitting underpants are worn.

2. The catheter-adapted, form fitting underpants of claim 1, wherein the clamp sewn to the underpants is sewn to fabric covering a wearer thigh when worn.

3. The catheter-adapted, form fitting underpants of claim 1, further comprising a diagonal functional fly relative to the horizontal.

4. The catheter-adapted underpants of claim 3, wherein the functional fly extends through a waistband band of the underpants so as to enable a catheter wearer to put on the underpants without disconnecting the catheter from a drainage bag.

5. The catheter-adapted of claim 4, wherein the functional fly is releasably closed through a hook-and-eye fastener.

6. The catheter-adapted underpants of claim 5, wherein the functional fly is releasably through a velcro patch and the hook-and-eye fastener simultaneously.

7. The catheter-adapted briefs of claim 1, wherein the catheter clamp encloses a catheter deployed in a clamp channel when the clamp is in a closed state.

8. (canceled)

9. The catheter-adapted underpants of claim 1, wherein the catheter clamp includes a polymeric material.

10. The catheter-adapted underpants of claim 1, further comprising a liquid absorbent lining.

11. The catheter-adapted underpants of claim 10, wherein the lining includes bamboo fiber.

12. The catheter-adapted underpants of claim 10, wherein the lining is implemented with anti-bacterial material.

13. The catheter-adapted underpants of claim 12, wherein the lining includes a copper-based material.

14. The catheter-adapted underpants of claim 1, further comprising an interfacing lining the underpants, the interfacing providing support to the catheter clamp.

15. The catheter-adapted underpants of claim 14, wherein the catheter port is disposed in a position vertically aligned with a natural anatomical position of the male urethral orifice when the underwear are worn.

16. Catheter-adapted underpants comprising:
a washable pair of elastic form fitting underpants; and
a catheter clamp snapped to underwear fabric covering a wearer thigh when worn, wherein the clamp causes catheter tug to be diverted from a male urethral orifice and distributed the thigh through the form fitting underpants when worn.

17. (canceled)

18. The catheter-adapted underpants of claim 16, further comprising a diagonal functional fly relative to the horizontal.

19. Catheter-adapted underpants comprising:
a washable pair of elastic form fitting underpants;
a catheter clamp operative to secure a catheter deployed in a clamp attached to fabric of the underpants covering a wearer thigh when worn; and
a connection means for connecting the catheter clamp to fabric of the underpants covering a wearer thigh when worn so as to distribute catheter tug to the wearer thigh when worn.

20. The catheter-adapted underpants of claim 19, further comprising a comprising a diagonal functional fly relative to the horizontal extending through a waistband band of the underpants.

21. The catheter adapted underpants of claim 20, wherein the functional fly is releasably closed through a velcro patch disposed on a waistband.

22. The catheter adapted underpants of claim 21, wherein the functional fly is releasably closed through a hook-and-eye fastener and the velcro patch simultaneously.
